(19) United States
(12) Patent Application Publication
(10) Pub. No.: US 2019/0328656 A1
(43) Pub. Date: Oct. 31, 2019

(54) TARGETED DELIVERY OF PROGESTINS AND ESTROGENS VIA VAGINAL RING DEVICES FOR FERTILITY CONTROL AND HRT PRODUCTS

(71) Applicant: Evestra, Inc., Schertz, TX (US)

(72) Inventors: Walter Elger, Berlin (DE); Klaus Nickisch, Berlin (DE); Ze'ev Shaked, San Antonio, TX (US); Karin Eggenreich, Schertz, TX (US); Simone Eder, Graz (AT); Andreas Witschnigg, Villach (AT)

(21) Appl. No.: 16/395,342

(22) Filed: Apr. 26, 2019

Related U.S. Application Data
(60) Provisional application No. 62/663,584, filed on Apr. 27, 2018.

(51) Int. Cl. A61K 9/00; A61P 15/18; A61K 31/575; A61K 31/567; A61K 31/565; A61K 47/32

(52) U.S. Cl. CPC A61K 9/0036; A61P 15/18; A61K 47/32; A61K 31/567; A61K 31/565; A61K 31/575

(57) ABSTRACT

A variety of different intravaginal drug delivery devices are described for the delivery of estrogens and progestins. The release rate of estrogens and progestins can be controlled by varying the matrix material or by the application of a thin coating. The intravaginal drug delivery devices may be composed of one or more individual compartments. By controlling various physical and chemical parameters, non-zero release rates of the estrogen or progestins may be achieved.

### PRIORITY CLAIM

This application claims priority to U.S. Provisional Application Ser. No. 62/663,584 entitled “TARGETED DELIVERY OF PROGESTINS AND ESTROGENS VIA VAGINAL RING DEVICES FOR FERTILITY CONTROL AND HRT PRODUCTS” filed Apr. 27, 2018, which is incorporated herein by reference in its entirety.

### BACKGROUND OF THE INVENTION

### 1. Field of the Invention

The present invention generally relates to drug delivery systems. More particularly, the invention relates to vaginal drug delivery systems, which release either an estrogen or a progestin alone or in combination according to an optimal pharmacological profile over a prolonged period of time.

### 2. Description of the Relevant Art

IVRs (intravaginal rings) are designed to release one or two hormones in the vaginal tract and eventually to deliver them into the systemic circulation over extended time periods. Their concept is based on two principles: First, IVRs are composed of polymers, in which the hormones show a certain permeability yielding certain release rates. Second, the vaginal epithelium can permeate hormones, thus, introducing the active ingredient into systemic action.

Currently, there are six IVRs on the market. ESTRING and FEMRING are reservoir systems based on silicone, whereas PROGERING and FERTIRING are silicone based matrix systems. GINORING is a reservoir system based on ethylene vinyl acetate (EVA) as core material and TPU (polyurethane) releasing two hormones, i.e., etonogestrel and ethinyl estradiol. Devices of this type, however, do not provide pharmacologically acceptable and/or optimal release profiles.

NuvaRing® is a reservoir system built up of two different EVA co-polymer types. The core is based in an EVA with 28% VA and supersaturated with ENG (i.e., 11.7 mg), whereas the EE concentration is below its saturation solubility (i.e., 2.7 mg). An EVA with 9% VA serves as the skin material, the skin thickness is around 110 μm according to the product description. Due to its reservoir design, the skin modulates the drug release. Both, EE and ENG are continuously released over 21 days with average daily in-vitro release rates of 15 μg and 120 μg during day 2 and day 20. However, the release rates may be increased on day 1 and decreased on day 21. On day 1, the so-called burst effect is likely to occur: Hormones, dissolved in the core material, gradually migrate into the skin according to their diffusion coefficient. Thereby, a certain amount of hormones is located at/close to the IVR's surface and is immediately dissolved when placed into the dissolution medium. Thereby, increased release rates are observed. At late time points, the remaining hormone fraction in the core may be lowered yielding decreased release rates.

Improvement was sought by using other shapes or materials. A two-layered, one compartment vaginal ring made from silicon elastomer has been disclosed in European Patent No. 0 050 867, in which a silicone elastomer core is loaded with active substance surrounded by a non-loaded silicone elastomer layer which consists of two different compositions.

Another improvement was claimed in U.S. Pat. No. 4,292,965 which discloses a three layered compartment ring. This ring comprises of an inert silicone elastomer core encircled by a medicated silicone layer and a non-medicated silicone outer layer.

Drug delivery systems for vaginal use, and in particular vaginal rings, prepared using polyethylene vinyl acetate (EVA) copolymers are also known in the art (see e.g., van Laarhoven et al., Journal of Pharmaceutics 232 (2002): 163-173).

European Patent No. 0 876 815 describes a one compartment vaginal ring comprising of EVA copolymer core containing ethinyl estradiol and etonogestrel and a non-medicated EVA outer membrane, which controls the release rate of the active components. This device releases two or more active substances in an essentially constant ratio to one another over an extended period of time. This concept was further developed in U.S. Published Patent Application No. 2014/0302115 in which the basic concept remains the same, but by using certain EVA polymers, a higher stability at room temperature was reached.

Other concepts described include using drug delivery devices having three, instead of two layers, whereas at least two of the three layers consists of drugs, see e.g., U.S. Patent Application Publication Nos. 2012/0148655, 2014/0350488 and 2009/0081278. All these drug delivery devices release the active ingredients in an essentially constant release rate, where the rate of release is controlled via the outer layer.

Another approach to the above problem is described in U.S. Pat. Nos. 7,829,112; 7,833,545; 7,838,024 and 7,883,718 that describe drug delivery devices that have two or more unitary segments composed of a drug—permeable polymeric substance, where at least one of the segments has a pharmaceutically active agent. A special noteworthy property of the claimed devices is that they deliver the active pharmaceutical agents at a substantially zero-order rate and that the segments do not have a membrane.

In addition to single drug delivery, vaginal rings have been developed for the simultaneous release of more than one drug at the time. The vaginal rings described in U.S. Pat. Nos. 3,995,633 and 3,995,634 have separate reservoirs containing different active substances, wherein the reservoirs are arranged in holders. In U.S. Pat. No. 4,237,885 multi-reservoir devices are described in which spacers are used to divide a tube into portions. Each portion is filled with a different active ingredient in a silicone fluid. PCT Application No. WO 97/02015 discloses a two-compartment device wherein one compartment has a core, a medicated middle layer, and a non-medicated outer layer and a second compartment has a medicated core and a non-medicated outer layer.

A further improvement was disclosed in U.S. Pat. No. 5,989,581 for the simultaneous release of a progestin compound and an estrogen compound, reportedly in a fixed ratio over a prolonged period of time. This approach was further modified in PCT Application No. WO 2015/086491 which describes an intra-vaginal drug delivery system having a core covered by a skin. The core is composed of a first thermoplastic polymer and a first therapeutic agent, where the first therapeutic agent is dissolved in the first thermoplastic polymer. A skin surrounding the core is composed of a second thermoplastic polymer, wherein the first therapeutic agent is less permeable in the second thermoplastic polymer than in the first thermoplastic polymer. A second therapeutic agent is loaded in a portion of the skin.

However, like other devices the ones disclosed in U.S. Pat. No. 5,989,581 and WO 2015/086491 suffer from their own inherent limitations. In general, the release per unit time of a drug is determined by the solubility of the active substance in the outer layer of polymeric material and by the diffusion coefficient of the active substance in the membrane. This is especially relevant if the two pharmaceutical ingredients have significantly different physicochemical properties in general and especially when it comes to different diffusion coefficients.

One approach to overcome the limitations of the low solubility of certain drugs in the polymer used as reservoir is described in U.S. Pat. No. 5,788,980. Addition of fatty acid esters increases the solubility of estrogens (e.g., estradiol) and progestins. The increased solubility leads to a zero-order rate of delivery over a prolonged period of time.

Another approach was disclosed in U.S. Patent Application Publication No. 2014/0209100 which describes devices that include a reservoir of at least one vaginally delivered drug, wherein the reservoir is surrounded by a hydrophilic elastomer. Such devices are capable of releasing hydrophilic drugs at a substantially zero-order rate over extended period of times.

In summary, although a large number of device concepts have been described, all of them suffer from at least one of the following drawbacks: inability to adjust the release of multiple therapeutic components, difficulty or expensive manufacturing process, inability to meet required release criteria to achieve the optimal targeted therapeutic effect and lack of stability upon storage and transport. This is especially evident when it is desirable to release the pharmaceutical agents in a nonzero-order kinetic fashion and when it is intended to release very hydrophilic compounds like estriol or highly active compounds like trimegestone.

In addition, notwithstanding the widespread use of estrogens in hormonal contraceptives, there are still some unresolved problems. Known estrogens, in particular the biogenic estrogens are eliminated from the blood stream very quickly. For instance, for the main biogenic estrogen 17-beta estradiol the half-life is around 1 hour. As a result, between separate administration events, blood serum levels of such biogenic estrogens tend to fluctuate considerably.

17a-ethinyl estradiol (EE) on the other hand, is still the leading estrogenic substance in the combined hormonal contraception. EE is contained in the leading oral and non-oral contraceptive products. It is used in the contraceptive vaginal ring (e.g., the Nuvaring®) and contraceptive transdermal patches. The liver is a target organ for estrogens. The secretion activity that is affected by estrogens in the human liver includes increased synthesis of transport proteins CBG, SHBG, TBG, several factors that are important for the physiology of blood clotting, and lipoproteins. The strong hepatic estrogenicity of ethinyl estradiol, especially their effects on hemostasis factors, may explain why these synthetic estrogens have been associated with the enhanced risk of thromboembolism. Other undesirable side-effects of synthetic estrogens include fluid retention, nausea, bloating, headache and breast pain.

The aforementioned deficits of synthetic estrogens are of considerable significance and consequently there is a significant unmet medical need for estrogens that do not display these deficits and which can suitably be employed in contraceptive methods for females because of their ability to reliably suppress follicle maturation and effectively replace the endogenous ovarian secretion of estradiol.

Estriol is used for the local therapy of certain menopausal symptoms. In U.S. Patent Application Publication No. 2011/0086825, a topical formulation is described of progesterone, testosterone and estriol. PCT Publication No. WO 2009/000954 describes the use of low dose estriol for the treatment/prevention of vaginal atrophy. PCT Publication No. WO 2011/0312929 describes an estriol formulation with the capacity to self-limit the absorption of estriol for the treatment of urogenital atrophy, and in PCT Publication No. WO 2010/069621 the treatment of vaginal atrophy for women with a cardiovascular risk is described.

A film based estriol oral formulation for the buccal application of estriol is described in PCT Publication No. WO 2005/110358 by Elger et al. for the treatment of climacteric symptoms. The same group describes in U.S. Pat. No. 5,614,213 a transdermal product that releases estriol over 24 hours. Estriol derivatives have been described. In U.S. Pat. No. 4,780,460, glycol esters of estriol have been described in order to form an aqueous crystalline suspension. In U.S. Pat. No. 4,681,875, 3,17-estriol esters were disclosed for the prolonged subcutaneous application of estriol. Estriol esters were also disclosed in U.S. Pat. No. 6,894,038 for the treatment of autoimmune diseases such as multiple sclerosis.

It can be concluded that no approach has been described to generate long-lasting therapeutic plasma levels of estriol that would be needed in order to treat climacteric symptoms and to provide activity in the prevention of osteoporosis.

Another approach to overcome the hepatic estrogenicity problem of ethinyl estradiol is disclosed in PCT Publication No. WO 02/094278 describing the use of estetrol as estrogenic component. Estetrol exhibits very weak estrogenic activity compared to estriol what leads to very high doses of 15 mg/day to reach pharmacological effects. Such high doses are a prohibitive for the development of innovative drug delivery forms like patches and or vaginal rings.

Use of trimegestone as a contraceptive agent in different applications has been described. In PCT Publication No. WO 03/084521 the use in combination with estradiol has been claimed as treatment for vasomotor symptoms. The contraceptive use, as an oral formulation has been described in PCT Publication No. WO 01/37841 and European Publication No. 0917466. Special drug delivery options have been claimed for patches in PCT Publication No. WO 9747333 and French Patent No. 2749586 and in form of vaginal rings for the HRT indication in European Publication No. 0 917 466.

In summary it can be concluded that, although it is quite obvious to experts in the field that a contraceptive product based on the natural estrogen estriol and trimegestone would be a very desirable product because of the lack of hepatic estrogenicity, no such product has been described so far, caused by the very low bioavailability and high renal clearance.

### SUMMARY OF THE INVENTION

In an embodiment, an intravaginal drug delivery device includes: one or more compartments, each of the compartments comprising an estrogen and/or a progestin dispersed in a thermoplastic polymeric matrix. The thermoplastic matrix is selected such that the intravaginal drug delivery device provides the estrogen and/or the progestin according to a non-zero order release profile.

In one embodiment, the intravaginal drug delivery device includes one or more uncoated compartments, where the uncoated compartments include an estrogen and/or progestin dispersed in an uncoated thermoplastic polymeric matrix. The intravaginal drug delivery device may also include one or more coated compartments, where the coated compartments include an estrogen and/or progestin dispersed in a coated thermoplastic polymeric matrix. The coated thermoplastic polymeric matrix includes a coating surrounding a thermoplastic polymeric matrix. The compartments, in some embodiments, have different sizes.

In an embodiment, the device includes at least one compartment containing a progestin. The progestin may be etonogestrel and/or trimegestone. In an embodiment, the device includes at least one compartment containing an estrogen. The estrogen may be ethinyl estradiol or estriol.

In an embodiment, the device releases trimegestone in doses between 0.05 and 0.5 mg/day. In an embodiment, the device releases estriol in doses between 0.05 and 0.8 mg/day. In an embodiment, the device's release of estriol such that estriol plasma levels of 50-200 pg/ml, on day 1 of treatment is achieved. In an embodiment, the device's release of estriol is such that estriol plasma levels of 15-30 pg/ml, on day 21 of treatment, is achieved.

In an embodiment, the device includes at least one compartment containing estriol. The estriol is released, during use, in amounts sufficient to treat vasomotor symptoms of postmenopausal women. In an embodiment, the device includes at least one compartment containing a progestin. The progestin is released, during use, in amounts sufficient to inhibit ovulation in fertile women. In an embodiment, the device includes at least one compartment containing an estrogen and at least one compartment containing a progestin. The estrogen and progestin are released, substantially simultaneously, during use, in amounts sufficient for ensuring good cycle control in fertile women.

In an embodiment, the thermoplastic matrix includes an ethylene vinyl acetate copolymer. In an embodiment, the thermoplastic matrix includes one or more hydrophilic matrix materials. In an embodiment, the thermoplastic matrix comprises an ethyl vinyl acetate copolymer and one or more hydrophilic matrix materials. In an embodiment, the device has a substantially annular form.

In an embodiment, the device delivers an effective amount of a progestin and an estrogen for at least 21 days. In an embodiment, the progestin and/or estrogen are released in a non-zero order fashion. A non-zero order release, in one embodiment, means that the ratio of the release rates of estriol and trimegestone on day 1 and day 21 are between 1.5 and 4.0. Alternatively, a non-zero order release means that the ratio of the release rates of estriol and trimegestone on day 1 and day 21 are between 1.5 and 3.0. Alternatively, a non-zero order release means that the ratio of the release rates of estriol and trimegestone on day 1 and day 21 are between 1.5 and 2.0.

In an embodiment, a method of producing a contraceptive state in a subject includes positioning an intravaginal drug delivery device, as described above, in the vagina or uterus of a female.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1A depicts an in-vitro release profile of ethinyl estradiol from a reservoir type intravaginal ring;

FIG. 1B depicts an in-vitro release profile of etonogestrel from a reservoir type intravaginal ring;

FIG. 2A depicts an in-vitro release profile of ethinyl estradiol from a matrix type intravaginal ring;

FIG. 2B depicts an in-vitro release profile of etonogestrel from a matrix type intravaginal ring;

FIG. 3A depicts an in-vitro release profile of trimegestone from a matrix type intravaginal ring with 0.25% loading;

FIG. 3B depicts an in-vitro release profile of trimegestone from a matrix type intravaginal ring with 0.50% loading;

FIG. 4A depicts an in-vitro release profile of trimegestone from a reservoir type intravaginal ring with 1.053% core loading and 320 μm skin thickness;

FIG. 4B depicts an in-vitro release profile of trimegestone from a reservoir type intravaginal ring with 1.053% core loading and 190 μm skin thickness;

FIG. 4C depicts an in-vitro release profile of trimegestone from a reservoir type intravaginal ring with 0.90% core loading and 135 μm skin thickness;

FIG. 5A depicts an in-vitro release profile of estriol from a matrix type intravaginal ring with 0.65% loading;

FIG. 5B depicts an in-vitro release profile of estriol from a matrix type intravaginal ring with 5% loading;

FIG. 5C depicts an in-vitro release profile of estriol from a matrix type intravaginal ring with 15% loading;

FIG. 5D depicts an in-vitro release profile of estriol from a matrix type intravaginal ring with 30% loading;

FIG. 6A depicts an in-vitro release profile of estriol from a matrix system of a segmented intravaginal ring (60% estriol loaded matrix segment length);

FIG. 6B depicts an in-vitro release profile of trimegestone from a reservoir system of a segmented intravaginal ring (1.053% core loading; 190 μm skin thickness; 40% trimegestone segment length);

FIG. 7 depicts a graph showing the concentration of Follicle Stimulating Hormone (FSH) for an etonogestrel and ethinyl estradiol containing intravaginal ring;

FIG. 8A depicts a graph showing mean plasma concentrations of estriol after single dose application for three different devices having different estriol delivery rates;

FIG. 8B depicts a graph showing mean plasma concentration vs. time curves for changes from baseline of FSH for three different devices having different estriol delivery rates;

FIG. 8C depicts a graph showing mean maturation index by cell types (parabasal, intermediate and superficial) for three different devices having different estriol delivery rates;

FIG. 9A depicts a graph showing mean plasma concentrations of estriol after single dose application for three different devices having different estriol and trimegestone delivery rates;

FIG. 9B depicts a graph showing mean plasma concentrations of trimegestone after single dose application for three different devices having different estriol and trimegestone delivery rates; and

FIG. 9C depicts a graph showing bleeding profile for women treated with an intravaginal device delivering estriol and trimegestone.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Thus, for example, reference to “a progestin” includes one or more progestins.

As used herein, an “intravaginal device” refers to an object that provides for administration or application of an active agent to the vaginal and/or urogenital tract of a subject, including, e.g., the vagina, cervix, or uterus of a female.

In an embodiment, an intravaginal drug delivery device includes one or two or more compartments joined to each other. Each of the compartments includes an estrogen and/or a progestin. Each compartment may be an uncoated polymeric matrix that includes the active agent or a coated polymeric matrix that includes the active agent. A combination of coated and uncoated compartments may be combined to form a ring-shaped drug delivery device.

A variety of materials may be used as the matrix for the compartments. Generally, the compartments used in the intravaginal device are suitable for extended placement in the vaginal tract or the uterus. In an embodiment, a thermoplastic material is used to form the intravaginal drug delivery device. The thermoplastic material is nontoxic and non-absorbable in the subject. In some embodiments, the materials may be suitably shaped and have a flexibility allowing for intravaginal administration.

In a preferred embodiment, compartments of an intravaginal drug delivery device are formed from an ethylene vinyl acetate copolymer (EVA). A variety of grades may be used including grades having a low melt flow index, a high melt flow index, a low vinyl acetate content or a high vinyl acetate content. As used herein, EVA having a “low melt flow index” has a melt flow index of less than about 100 g/10 min as measured using ASTM test 1238. EVA having a “high melt flow index” has a melt index of greater than about 100 g/10 min as measured using ASTM test 1238. EVA having a “low vinyl acetate content” has a vinyl acetate content of less than about 20% by weight. EVA having a “high vinyl acetate content” has a vinyl acetate content of greater than about 20% by weight. The compartments of the intravaginal drug delivery device may be formed from EVA having a low melt flow index, a high melt flow index, a low vinyl acetate content or a high vinyl acetate content. In some embodiments, the thermoplastic matrix may include: mixtures of a low melt flow index and high melt flow index EVA or mixtures of low vinyl acetate content and high vinyl acetate content EVA.

In an embodiment, a combination of one or more suitable materials may be used to form the compartments. The material(s) may be selected to allow prolonged release of the active ingredients from the compartment. In addition, the concentration of the active agents, in combination with the matrix material may be selected to provide the desired release from the compartment. In some compartments, a coating may be applied to the matrix to yield reservoir systems to further control the release rate of the active ingredients. The coating may be formed from the same material, or a different material than the thermoplastic matrix used to form the compartment.

In one embodiment, the compartment may be composed of ethylene vinyl acetate copolymer in combination with the hydrophobic polymer hydroxy propyl cellulose.

In an embodiment, the active agents, for example the progestin and/or estrogen, are dispersed in the thermoplastic matrix to form a compartment. As used herein the term “dispersed”, with respect to a thermoplastic matrix, means that a compound is substantially evenly distributed through the polymer, either as a solid dispersion in the polymer or dissolved within the polymer matrix. The term “particle dispersion,” as used herein refers to a dispersion of the compound particles homogenously distributed in the polymer. The term “molecular dispersion,” as used herein refers to the dissolution of the compound in the polymer. For purposes of this disclosure, a dispersion may be characterized as a particle dispersion if particles of the compound are visible in the polymer at a magnification of about 100-fold under regular and polarized light. A molecular dispersion is characterized as a dispersion in which substantially no particles of the compound are visible in the polymer at a magnification of 100-fold under regular and polarized light.

In an embodiment, the intravaginal drug delivery device is used to produce a contraceptive state in a female mammal. The contraceptive state may be produced by administering an intravaginal drug delivery device that includes a progestin. In other embodiments, contraceptive state may be produced by administering an intravaginal drug delivery device that includes a progestin and an estrogen component.

Progestins include, but are not limited to, gestodene, ketodesogestrel, demegetone, desogestrel, drospirenone, levonorgestrel, megestrol, megestrol acetate, melengestrol, melengestrol acetate, nestorone, nomegestrol acetate, norgestimate, and promegestone.

A preferred progestin is trimegestone and a preferred estrogen is estriol.

The intravaginal delivery device can be in any shape suitable for insertion and retention in the vaginal tract without causing undue discomfort to the user. For example, the intravaginal device may be flexible. As used herein, “flexible” refers to the ability of an intravaginal drug delivery device to bend or withstand stress and strain without being damaged or broken. For example, an intravaginal delivery device may be deformed or flexed, such as, for example, using finger pressure, and upon removal of the pressure, return to its original shape. The flexible properties of the intravaginal drug delivery device are useful for enhancing user comfort, and also for ease of administration to the vaginal tract and/or removal of the device from the vaginal tract.

In an embodiment, the intravaginal drug delivery device may be annular in shape. As used herein, “annular” refers to a shape of, relating to, or forming a ring. Annular shapes suitable for use include a ring, an oval, an ellipse, a toroid, and the like. The intravaginal drug delivery device may have a non-annular geometry.

In one embodiment, the intravaginal drug delivery device has a geometry in the form of a strand of geometrically shaped compartments linked together. For example, a plurality of hexagon shaped compartments may be linked to form a strand. Other geometrically shaped units including, but not limited to, squares, triangles, rectangles, pentagons, heptagons, octagons, etc. may be formed into strands. In some embodiment, mixtures of different geometrically shaped units may be joined to together in a strand. The strand of geometrically shaped units may be joined together to form ring-like structure.

In another embodiment, an intravaginal drug delivery device is in the shape of a half oval. A half oval device may be easier to manufacture than a full ring. In an embodiment, the half oval shape may allow a user to form a ring like structure before and/or after insertion. In another embodiment, an intravaginal drug delivery device may be in the shape of a hollow cylinder. Use of a hollow cylinder may allow easier insertion of the intravaginal delivery device. The hollow cylinder geometry may allow insertion of the intravaginal drug delivery device into the vaginal tract in a compressed form, which, upon deployment, expands inside the tract to improve the retention of the device. In another embodiment, an intravaginal drug delivery device may have a monolithic film geometry. Such a film may be formed or include, mucoadhesive substances to improve adhesion to the vaginal tract.

The intravaginal drug delivery device may be manufactured by any known techniques. In some embodiments, therapeutically active agent(s) may be mixed within the thermoplastic matrix material and processed to the desired shape by: injection molding, rotation/injection molding, casting, extrusion, or other appropriate methods. In one embodiment, the intravaginal drug delivery device is produced by a hot-melt extrusion process.

In one embodiment, a method of making an intravaginal drug delivery device includes:
a. forming a mixture of a thermoplastic polymer and the active agent;
b. heating the thermoplastic polymer/active agent mixture such that at least a portion of the thermoplastic polymer is softened or melted to form a heated mixture of thermoplastic polymer and active ingredient;
c. permitting the heated mixture to cool and solidify as a solid mass,
d. and optionally, shaping the mass into a predetermined geometry.

For purposes of the present disclosure a mixture is “softened” or “melted” by applying thermal or mechanical energy sufficient to render the mixture partially or substantially completely molten. For instance, in a mixture that includes a matrix material, “melting” the mixture may include substantially melting the matrix material without substantially melting one or more other materials present in the mixture (e.g., the therapeutic agent and one or more excipients). For polymers, a “softened” or “melted” polymer is a polymer that is heated to a temperature at or above the glass transition temperature of the polymer. Generally, a mixture is sufficiently melted or softened, when it can be extruded as a continuous rod, or when it can be subjected to injection molding.

The mixture of the thermoplastic polymer and the active agent can be produced using any suitable means. Well-known mixing means known to those skilled in the art include dry mixing, dry granulation, wet granulation, melt granulation, high shear mixing, and low shear mixing.

Granulation generally is the process wherein particles of powder are made to adhere to one another to form granules, typically in the size range of 0.2 to 4.0 mm. Granulation is desirable in pharmaceutical formulations because it produces relatively homogeneous mixing of different sized particles.

Dry granulation involves aggregating powders with high compressional loads. Wet granulation involves forming granules using a granulating fluid including either water, a solvent such as alcohol or water/solvent blend, where this solvent agent is subsequently removed by drying. Melt granulation is a process in which powders are transformed into solid aggregates or agglomerates while being heated. It is similar to wet granulation except that a binder acts as a wetting agent only after it has melted. The granulation is further achieved following using milling and/or sieving to obtain the desired particle sizes or ranges. All of these and other methods of mixing pharmaceutical formulations are well-known in the art.

Subsequent or simultaneous with mixing, the mixture of thermoplastic polymer and the active agent is softened or melted to produce a mass sufficiently fluid to permit shaping of the mixture and/or to produce melding of the components of the mixture. The softened or melted mixture is then permitted to solidify as a substantially solid mass. The mixture can optionally be shaped or cut into suitable sizes during the softening or melting step or during the solidifying step. In some embodiments, the mixture becomes a homogeneous mixture either prior to or during the softening or melting step. Methods of melting and molding the mixture include, but are not limited to, hot-melt extrusion, injection molding and compression molding.

Hot-melt extrusion typically involves the use of an extruder device. Such devices are well-known in the art. Such systems include mechanisms for heating the mixture to an appropriate temperature and forcing the melted feed material under pressure through a die to produce a rod, sheet or other desired shape of constant cross-section. Subsequent to or simultaneous with being forced through the die the extrudate can be cut into smaller sizes appropriate for use as an oral dosage form. Any suitable cutting device known to those skilled in the art can be used, and the mixture can be cut into appropriate sizes either while still at least somewhat soft or after the extrudate has solidified. The extrudate may be cut, ground or otherwise shaped to a shape and size appropriate to the desired oral dosage form prior to solidification, or may be cut, ground or otherwise shaped after solidification. In some embodiments, an oral dosage form may be made as a non-compressed hot-melt extrudate. In other embodiments, an oral dosage form is not in the form of a compressed tablet.

Injection molding typically involves the use of an injection-molding device. Such devices are well-known in the art. Injection molding systems force a melted mixture into a mold of an appropriate size and shape. The mixture solidifies as least partially within the mold and then is released.

Compression molding typically involves the use of a compression-molding device. Such devices are well-known in the art. Compression molding is a method in which the mixture is optionally preheated and then placed into a heated mold cavity. The mold is closed and pressure is applied. Heat and pressure are typically applied until the molding material is cured. The molded oral dosage form is then released from the mold.

The final step in the process of making intravaginal drug delivery device is permitting the mixture to solidify as a solid mass. The mixture may optionally be shaped either prior to solidification or after solidification. Solidification will generally occur either as a result of cooling of the melted mixture by different methods (air, water bath) or as a result of curing of the mixture however any suitable method for producing a solid dosage form may be used.

When combining compartments to form an intravaginal drug delivery device, individual compartments may be joined directly together or may be coupled to each other through a spacer formed form a thermoplastic matrix material. The spacer may be formed from the same thermoplastic material used to form the compartments, or may be formed from a different material. The spacer, in some embodiments, does not include any active agents.

Through the use of different compartments in the drug delivery device, the device releases the active ingredients such that each of the released active ingredients has a different non-zero order release kinetic profile, and the amounts of active ingredients released are not constant but rather changing over time. Such release profiles are especially useful in the field of contraception and menopause management.

In one embodiment, a combination of compartments is selected to create release profiles that mimic hormone profiles of regular female cycle, with estrogen being more dominate in the first half, and progestin being more dominate in the second half of the cycle. In some embodiments, compartments may be selected to enable delivery of high concentrations of a progestin, which is responsible for ovulation inhibition, from the first day of treatment to avoid further growth of the leading follicle that has grown in the hormone free interval between two cycles. The timing of the delivery of the appropriate amounts of progestin with the appropriate estrogen ensures a good bleeding profile.

In another preferred embodiment the estrogen is estriol and the progestin is trimegestone. In another preferred embodiment the estrogen is ethinyl estradiol and the progestin is etonogestrel.

Since estriol is a natural estrogen, its use is especially desirable since it offers significant advantages over synthetic estrogens (e.g., ethinyl estradiol and estradiol) when it comes to safety in indications like contraception and menopause management. Some of the advantages of estriol are: (a) lack of hepatic estrogenicity; (b) no stimulatory effect on breast tissue; (c) less induction of bleeding episodes than estradiol in postmenopausal women.

Estriol, however, offers a significant challenge when it comes to securing therapeutic plasma levels over the whole cycle based on the short half-life, the low solubility in thermoplastic polymers and the high doses that need to be delivered daily based on the lower intrinsic activity of estriol compared to estradiol and ethinyl estradiol. There are just three vaginal ring products releasing estrogenic compounds on the market: NUVARING, releasing 0.015 mg ethinyl estradiol per day; FEMRING, releasing 0.0075 mg estradiol per day; and ESTRING, releasing 0.05 to 0.1 mg estradiol acetate per day. It is noteworthy to mention, that for accomplishing a daily release of 0.1 mg estradiol, the ESTRING device uses a more lipophilic prodrug of estradiol, namely the estradiol 3-acetate.

In one embodiment, an intravaginal drug delivery system includes one or more compartments, each of the compartments including progestin and/or estrogen embedded in a thermoplastic polyethylene vinyl acetate copolymer. The progestin and/or estrogen may be either fully dissolved or in a crystalline stage. Each compartment may be an uncoated matrix of thermoplastic polyethylene vinyl acetate copolymer with the active agent(s) dispersed throughout the core. In some embodiments, a compartment may be a coated matrix having a thermoplastic polyethylene vinyl acetate copolymer covering the core.

The individual compartments, may be welded together to form a ring shaped drug delivery system by using a thermoplastic polymer spacer to link the compartments together. The spacers may be formed from a polyethylene vinyl acetate copolymer capable of inhibiting the exchange of estrogens and progestins from one compartment to the other.

One significant advantage of the intravaginal drug delivery devices described herein is that targeted release profiles can be generated by either: varying the size of the compartments (e.g., the length); varying the loading of active agents (e.g., the progestin or estrogen); adding a coating material to the compartment; or using a combination of any of these modifications.

Release kinetics identify the drug release process via mathematical models to drug release process (the amount of drug release per unit time). Release kinetics can also be defined by the ratio of active agent released on Day 1 to active agent release on the last day of administration (Day 21 or Day 28). For supersaturated systems where co (initial concentration at to) is above the cₛ (saturation concentration), release can also be fitted using the Korsmeyer-Peppas equation, where the drug fraction dissolved at a time, equivalent to active agent release, as a function of time is plotted. The diffusional exponent “n” of the power law and thereby, the drug release mechanism from different polymeric controlled delivery systems for different geometries (thin films, spheres or cylinders) can be determined via the slope of the linear regression fit. The release kinetics follows zero order release (Case-II transport), when the drug release is constant over time (ratio of releases Day 1 to Day 28 is 1) and independent of concentration. For cylinders, a diffusional exponent n of 0.89 or above indicates Case-II Transport and hence, zero order release.

The target release kinetics of a non-zero order release is provided for Day 1/Day 21 (or Day 28) ratios between 1.5 and 4.0. In the Korsmeyer-Peppas equation, non-zero order or anomalous transport (a combination of Case-II transport and Fickian diffusion) is achieved when the diffusional exponent n is between 0.89 and 0.45. A diffusional exponent of 0.45 indicates Fickian diffusion.

In preferred embodiments, the compartments include an active agent as a substantially uniform dispersion within a thermoplastic matrix. In alternative embodiments the distribution of the active agent within the thermoplastic matrix can be substantially non-uniform. One method of producing a non-uniform distribution of the active agent is through the use of one or more coatings of water-insoluble or water-soluble polymers. Another method is by providing two or more mixtures of polymer or polymer and the active agent to different zones of a compression or injection mold. These methods are provided by way of example and are not exclusive.

In practice, for a human female, an annular intravaginal drug delivery device has an outer ring diameter from 35 mm to 70 mm, from 35 mm to 60 mm, from 45 mm to 65 mm, or from 50 mm to 60 mm. The cross-sectional diameter may be from 1 mm to 10 mm, from 2 mm to 6 mm, from 3.0 mm to 5.5 mm, from 3.5 mm to 4.5 mm, or from 4.0 mm to 5.0 mm.

The release rate can be measured in vitro using compendial methods, e.g., the USP Apparatus Paddle 2 method, or a rotational incubation shaker. The active agent(s) can be assayed by methods known in the art, e.g., by HPLC or UPLC.

In some embodiments of the present invention, active agent(s) is/are released from the intravaginal device for up to about 1 month or about 28 days after administration to a female, for up to about 25 days after administration to a female, for up to about 21 days after administration to a female, for up to about 15 days after administration to a female, for up to about 10 days after administration to a female, for up to about 7 days after administration to a female, or for up to about 4 days after administration to a female.

Each individual compartment may release an active agent at a steady rate. As used herein, a “steady rate” is a release rate that does not vary by an amount greater than 70% of the amount of active agent released per 24 hours in situ, by an amount greater than 60% of the amount of active agent released per 24 hours in situ, by an amount greater than 50% of the amount of active agent released per 24 hours in situ, by an amount greater than 40% of the amount of active agent released per 24 hours in situ, by an amount greater than 30% of the amount of active agent released per 24 hours in situ, by an amount greater than 20% of the amount of active agent released per 24 hours in situ, by an amount greater than 10% of the amount of active agent released per 24 hours in situ, or by an amount greater than 5% of the amount of active agent released per 24 hours in situ.

In some embodiments, the active agent is trimegestone with a compartment steady release rate of active agent in situ of about 80 μg to about 200 μg per 24 hours, about 90 μg to about 150 μg per 24 hours, about 90 μg to about 125 μg per 24 hours, or about 95 μg to about 120 μg per 24 hours.

In some embodiments, the active agent is estriol with a compartment steady release rate of active agent in situ of about 50 μg to about 800 μg per 24 hours, about 100 μg to about 500 μg per 24 hours, about 150 μg to about 300 μg per 24 hours.

The release kinetics and drug release profile can be impacted by selecting the type of system. Reservoir systems are designed to yield zero order release kinetics (Case-II transport), whereas matrix systems provide either Fickian diffusion (drug release proportional to surface and drug loading) or anomalous transport (combination of Fickian diffusion and Case-II transport). For reservoir systems, release rates can be modulated by the skin thickness and type of polymer used. EVA copolymers with high vinyl acetate (VA) content show reduced crystallinity and hence, increased permeability, whereas EVA polymers with low VA content yield increased crystallinity and hence, reduced permeability.

In some embodiments, the active agent is released according to a non-zero order release, where the ratio of active agent release Day 1 to Day 21/28 is in the range of 1.5-4.0, more specifically, the ratio is in the range of 1.5-3.0, even more specifically, in the range of 1.5-2.0.

In some embodiments, the active agent is released according to anomalous transport (a combination of Case-II transport and Fickian diffusion). This refers to a diffusional exponent (in the Korsmeyer-Peppas Equation) for cylinders of 0.89-0.45.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Vaginal Ring Releasing Etonogestrel and Ethinyl Estradiol, Reservoir System

### Premix Preparation:

Ethinyl estradiol and etonogestrel loaded powder blends are prepared by dry blending the active agents and the polymer ethylene vinyl acetate using different blending techniques (e.g., tumble blending) and blending parameters, yielding a powder blend where the active agent is homogeneously distributed in the blend.

### Co-Extrusion:

The ethinyl estradiol and etonogestrel loaded ethylene vinyl acetate is co-extruded at low throughput ranges of <3 kg/h using a twin screw extruder for the drug loaded core material and a single screw extruder for the drug free ethylene vinyl acetate with lower VA content. The target skin thickness of 110 μm can be achieved via single screw extruder speeds of <10 rpm. The obtained co-extrudate (reservoir system) is subsequently cooled to yield co-axial fibers with an outer diameter of 4.0 mm and a pre-defined skin thickness. The co-extrudate diameter and sphericity may be controlled in-line using a multiple laser head system.

Ring Closure:

The ethinyl estradiol and etonogestrel loaded reservoir strands are cut into segments of 154 mm either manually or using a semi-automated system prior to being shaped to the vaginal ring via a welding step (e.g., hot air welding, injection molding) inside a ring-shaped mold with a single or multiple cavities of 4.0 mm cross-sectional diameter and 54 mm outer diameter. As welding material, the drug free ethylene vinyl acetate, serving as the core polymer, is used. The obtained rings are then stored at 5° C.

### Example 2—Vaginal Ring Releasing Etonogestrel and Ethinyl Estradiol, Matrix-Matrix System

### Premix Production:

Etonogestrel and ethinyl estradiol loaded powder blends are mixed by dry blending 7 parts of ethinyl estradiol and 40 parts of etonogestrel and 953 parts of hydroxy propyl cellulose using different blending techniques (e.g., tumble blending) and blending parameters, yielding a powder blend where the active agents are homogeneously distributed in the blend.

### First Extrusion:

In a first extrusion step, the drug loaded cellulose powder blend is processed via hot melt extrusion using a twin screw extruder and subsequent cooling to yield strands with an outer diameter of around 2.5 mm, which are then pelletized via strand granulation to obtain drug loaded polymer pellets.

### Second Extrusion:

In a second extrusion step, the drug loaded cellulose-based polymer pellets are further processed via hot melt extrusion in a twin screw extruder with ethylene vinyl acetate. This can be achieved by either blending the drug loaded cellulose pellets with the ethylene vinyl acetate in a ratio of >90 parts EVA and <10 parts drug loaded cellulose pellets and subsequent hot melt extrusion or by simultaneous processing of the drug loaded pellets and the ethylene vinyl acetate via split feeding and hot melt extrusion to strands with an outer diameter of around 2.5 mm and subsequent granulation of the extruded strands.

### Injection Molding:

The ethinyl estradiol and etonogestrel loaded hydroxy propyl cellulose pellets, embedded into the ethylene vinyl acetate copolymer, are shaped via injection molding using a single or multiple cavity ring shaped mold to yield a ring shaped device of 4.0 mm cross-sectional diameter and 54 mm outer diameter. Sufficient tensile strength is obtained by applying optimized injection molding parameters.

### Washing Step:

A final washing step is applied to reduce the burst effect, i.e., the amount of active ingredients released during the first days of ring application via depleting the outer regions of the rings. As washing agent, different types of solvents or solvent mixtures may be applied. The extent of active ingredients washed out and hence, the extent of the burst effect is controlled by the different washing parameters (e.g., type and volume of washing agent, washing time, temperature). The washed rings are finally rinsed with water and subsequently dried prior to being packed into individual sachets.

### Example 3—Trimegestone Vaginal Ring, Matrix System

### Premix Preparation:

Trimegestone loaded powder blends containing 0.25% and 0.50% trimegestone are prepared by dry blending the active agent and ethylene vinyl acetate using different blending techniques (e.g., tumble blending) and blending parameters, yielding a powder blend where the active agent is homogeneously distributed in the blend.

### Extrusion:

In a matrix extrusion step, the drug loaded premix is processed via hot melt extrusion using a twin screw extruder. The melt temperature was around 100° C. The extrudate was subsequently cooled at ambient temperature to solidify the melt and yield drug loaded matrix strands of 4.0 mm outer diameter. The co-extrudate diameter and sphericity may be controlled in-line using a multiple laser head system.

### Ring Closure:

The drug loaded matrix fibers are cut into segments of 154 mm either manually or using a semi-automated system prior to being shaped to the vaginal ring via a welding step (e.g., hot air welding, injection molding) inside a ring-shaped mold with a single or multiple cavities of 4.0 mm cross-sectional diameter and 54 mm outer diameter. As welding material, the drug free ethylene vinyl acetate is used. The obtained rings are then stored at 5° C.

### Example 4—Trimegestone Vaginal Ring of Different Skin Thickness, Reservoir Systems

### Premix Preparation:

Trimegestone loaded powder blends containing identical loadings in the core (=1.053%) are prepared by dry blending the active agent and the polymer ethylene vinyl acetate using different blending techniques (e.g., tumble blending, high shear blender) and blending parameters, yielding a powder blend where the active agent is homogeneously distributed in the blend.

### Co-Extrusion:

The trimegestone loaded ethylene vinyl acetate is co-extruded using a twin screw extruder for the drug loaded core material and a single screw extruder for the drug free ethylene vinyl acetate with a lower VA content (12%). The single screw extruder speed is adjusted to screw speed in order to yield the target skin thickness. By running the single screw extruder at low screw speeds of <5 rpm, a skin thickness of 135 μm can be achieved. Doubling the single screw extruder speed produces a skin thickness of 190 μm, and by a further screw speed increase of above 20 rpm, an increased skin thickness of 320 μm can be produced. The obtained co-extrudate is subsequently cooled to yield co-axial fibers with an outer diameter of 4.0 mm and the distinct skin thicknesses of 135 μm, 190 μm and 320 μm. The co-extrudate diameter and sphericity may be controlled in-line using a multiple laser head system.

### Ring Closure:

The trimegestone loaded reservoir fibers are cut into segments of 154 mm either manually or using a semi-automated system prior to being shaped to the vaginal ring via a welding step (e.g., hot air welding, injection molding) inside a ring-shaped mold with a single or multiple cavities of 4.0 mm cross-sectional diameter and 54 mm outer diameter. As welding material, the drug free ethylene vinyl acetate, serving as the core polymer, is used. The obtained rings are then stored at 5° C.

### Example 5—Estriol Vaginal Ring, Matrix System

### Premix Preparation:

Estriol loaded powder blends of different loadings (in the range of 0.625% to 30% w/w) are prepared by dry blending the active agent and the high VA content ethylene vinyl acetate, using different blending techniques (e.g., tumble blending, active blending via high shear forces) and blending parameters, yielding a powder blend where the active agent is homogeneously distributed in the blend.

### Extrusion:

In a matrix extrusion step, the drug loaded premix is processed via hot melt extrusion using a twin screw extruder and subsequent cooling at ambient temperature to yield drug loaded matrix strands of 4.0 mm outer diameter. The temperature configuration is slightly adapted depending on the drug loading and hence, the resulting melt viscosity to achieve a stable extrusion process and spherical extrudates.

### Ring Closure:

The drug loaded matrix fibers are cut into segments of 154 mm either manually or using a semi-automated system prior to being shaped to the vaginal ring via a welding step (e.g., hot air welding, injection molding) inside a ring-shaped mold with a single or multiple cavities of 4.0 mm cross-sectional diameter and 54 mm outer diameter. As welding material, the drug free ethylene vinyl acetate is used. The obtained rings are then stored at 5° C.

### Example 6—Estriol/Trimegestone Vaginal Ring, Segmented (Matrix/Reservoir) System

Combining Estriol with Trimegestone Containing Segments and Ring Closure:

Estriol loaded segments, prepared according to Example 3, are cut into segments of 92 mm (60% of the full ring). Trimegestone containing co-extrudates of 190 μm, prepared according to Example 5, are cut into segments of 60 mm (corresponding to 40% of the full ring). Cutting is done either manually or using a semi-automated system. The two segments are then joined in 2 subsequent welding steps (e.g., hot air welding, injection molding) inside a ring-shaped mold with a single or multiple cavities to yield one or multiple vaginal rings of 4.0 mm cross-sectional diameter and 54 mm outer diameter. As welding material, the drug free ethylene vinyl acetate, serving as the carrier for the matrix and the core polymer for the reservoir system, is used. The welding material serves the purpose of forming a ring, but can also act as a barrier to prevent the active agents from migration. This is achieved by selecting polymers with reduced VA content or no VA such as LDPE, that show higher crystallinity and hence, a lower and/or no permeability. Thereby, the welding material can act as barrier to avoid diffusion of the active ingredient from one segment into the other. The obtained rings are then stored at 5° C.

### In Vitro Release Rates

### Methods

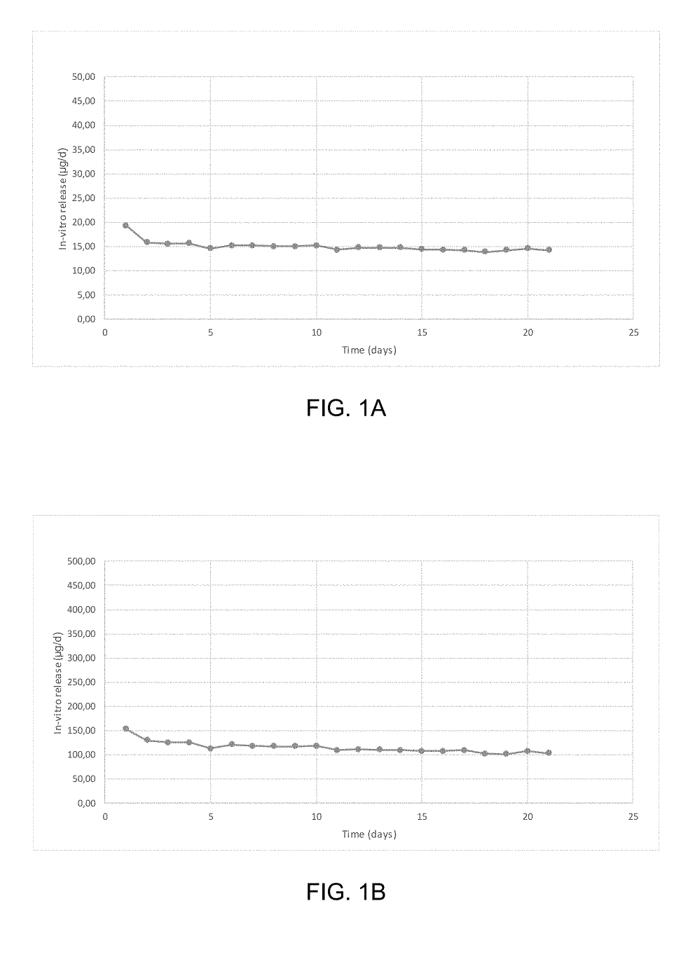

For in vitro dissolution testing, a rotational incubator operated at 37±0.5° C. is used. The type of dissolution medium, its volume and the incubator rotational speed are selected to provide sink conditions. Samples of 1 mL are withdrawn every 24±0.5 h (and multiples thereof) over 21 or 28 days, the medium is replaced every 24±0.5 h (and multiples thereof) by fresh media and the samples are analyzed for the drug content via (ultra) high performance liquid chromatography (UPLC/HPLC). The results of the tests on the rings of Examples 1-8 are depicted in FIGS. 1-8.

FIGS. 1A and 1B show the release profiles of a reservoir system, where etonogestrel is supersaturated. FIG. 1A gives the release rate of ethinyl estradiol during dissolution testing of the ring formed according to Example 1. FIG. 1B shows the release rate of etonogestrel during dissolution testing of the ring produced according to Example 1. The ratio of the ethinyl estradiol release rates day 1 to day 21 is 1.40, for etonogestrel, the ratio of the release rates d1/d21 is 1.50, indicating zero order release. Data fitting in the zero order release model yields a diffusional exponent n of 0.93 for ethinyl estradiol and 0.91 for etonogestrel, indicating zero order release rates.

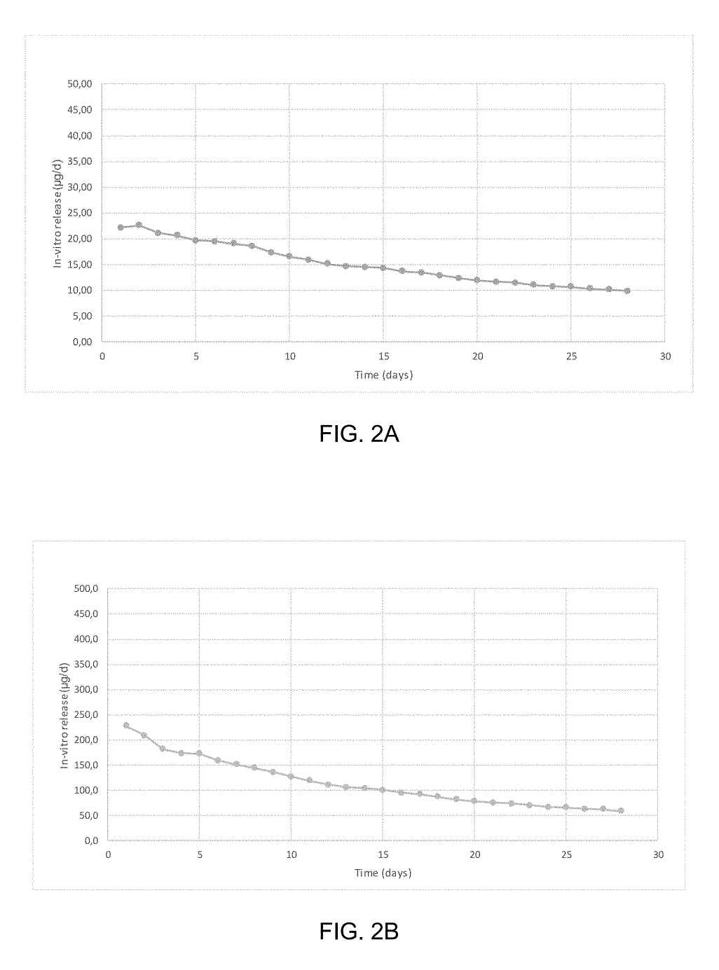

FIG. 2 shows the release rates of ethinyl estradiol and etonogestrel from a ring formed in Example 2, where the two actives are embedded in a hydrophilic carrier, which is further embedded in an EVA with high VA content. FIG. 2A shows the release rate of ethinyl estradiol during dissolution testing produced according to Example 2. FIG. 2B shows the dissolution profile of etonogestrel during dissolution testing of the ring formed according to Example 1, where etonogestrel is supersaturated in the core. The ratio of the ethinyl estradiol release rates on day 1 to day 21 is 1.90, for etonogestrel the ratio of the release rates d1/d21 is 3.03. Data fitting in the zero-order release model yielded diffusional exponents of n=0.89 for ethinyl estradiol, indicating zero-order release, and n=0.80 for etonogestrel.

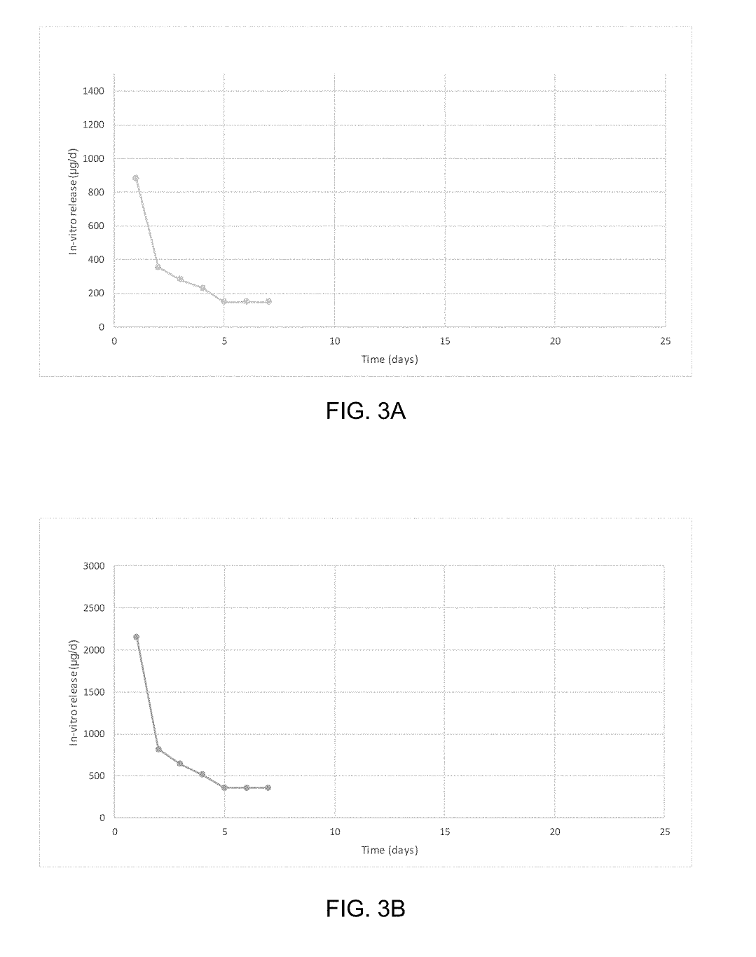

Generally, trimegestone can also be formulated into both, a matrix and a reservoir system. Matrix formulations containing trimegestone in an EVA carrier with high VA content according to Example 3 were tested with core loadings of 4.3 mg (=0.25%) and 8.6 mg (=0.50%). FIG. 3A shows the release rates of the matrix system with 0.25% trimegestone, FIG. 3B depicts the release of a 0.50% loaded matrix system.

The daily release of trimegestone increases with increasing drug loading. For both drug loadings, the release of trimegestone highly exceeds the target release values for the intended application, and more than 50% of the incorporated trimegestone is already released within one week, attributed to the formation of a solid dispersion comprising amorphous trimegestone, which is obviously highly diffusive in the EVA. The diffusional exponent n for this matrix systems is 0.37 and 0.47 for the 0.25% and 0.50% loadings, respectively. This suggests that the simple matrix approach is not applicable and a skin needs to introduced in order to tailor (i.e., decrease) the TMG release. The dissolution was therefore ended after 7 days. The diffusion coefficient of trimegestone is similar regardless of the VA content of the EVA polymer. Permeability of trimegestone is similar for EVA with high VA contents, but lower for EVA with low VA content (by a factor of 10). However, the solubilities are different and are significantly lower for low VA content EVAs, hence its permeability is decreased for lower VA contents due to its decreased solubility in the polymer, leading to reservoirs systems as delivery concept for trimegestone to achieve the target therapeutic release rates.

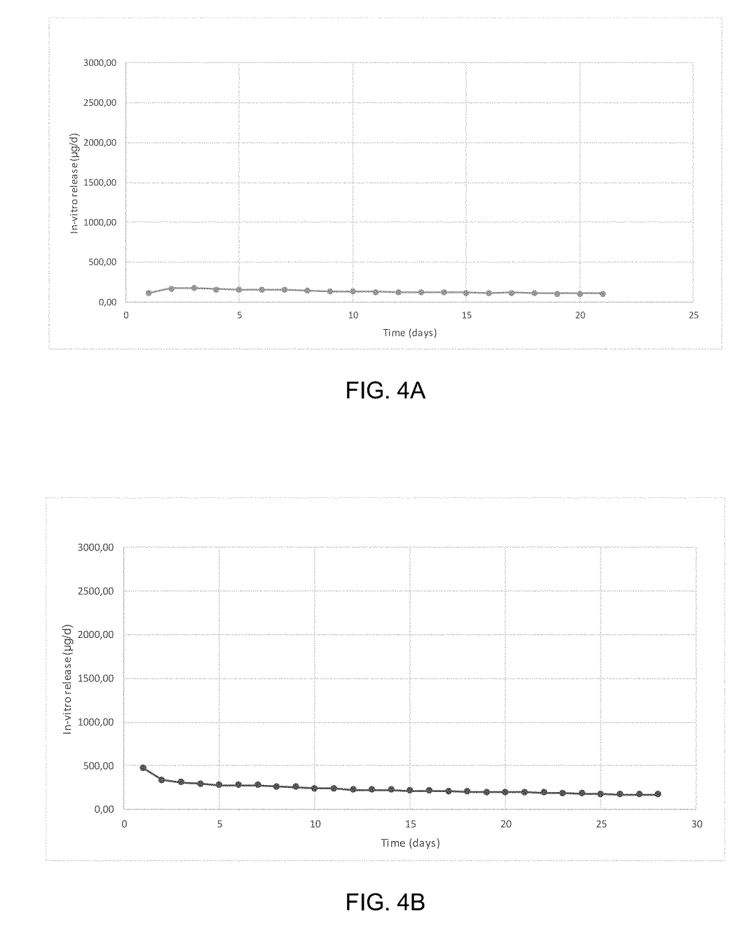

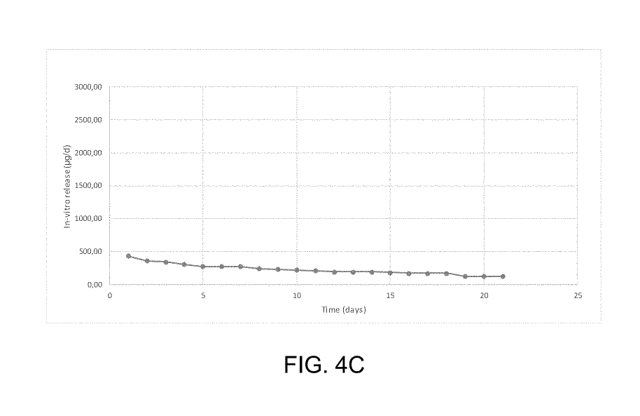

The release rates in the reservoir system can be modulated via the skin thickness. For low VA skin types and skin thicknesses of 190 μm and 320 μm, manufactured according to Example 4, zero order release was achieved with n=0.90 and d1/21 ratio of 1.62 for IVRs with 320 μm and n=0.89, d1/d21=2.43 for 190 μm skin thickness. FIG. 4A shows the release profile of a reservoir system (1.053% core loading according to Example 4) for a skin thickness of 320 μm, FIG. 4B depicts the release profiles of trimegestone for a reservoir IVRs (1.053% core loading) with 190 μm skin thickness.

When the skin thickness was decreased to 135 μm, data fitting showed anomalous transport (combination of Case-II and Fickian diffusion) as the underlying mechanism, with a diffusional exponent n of 0.76 for this formulation. FIG. 4C depicts the release profiles of trimegestone for a reservoir IVRs (1.053% core loading) with 135 μm skin thickness.

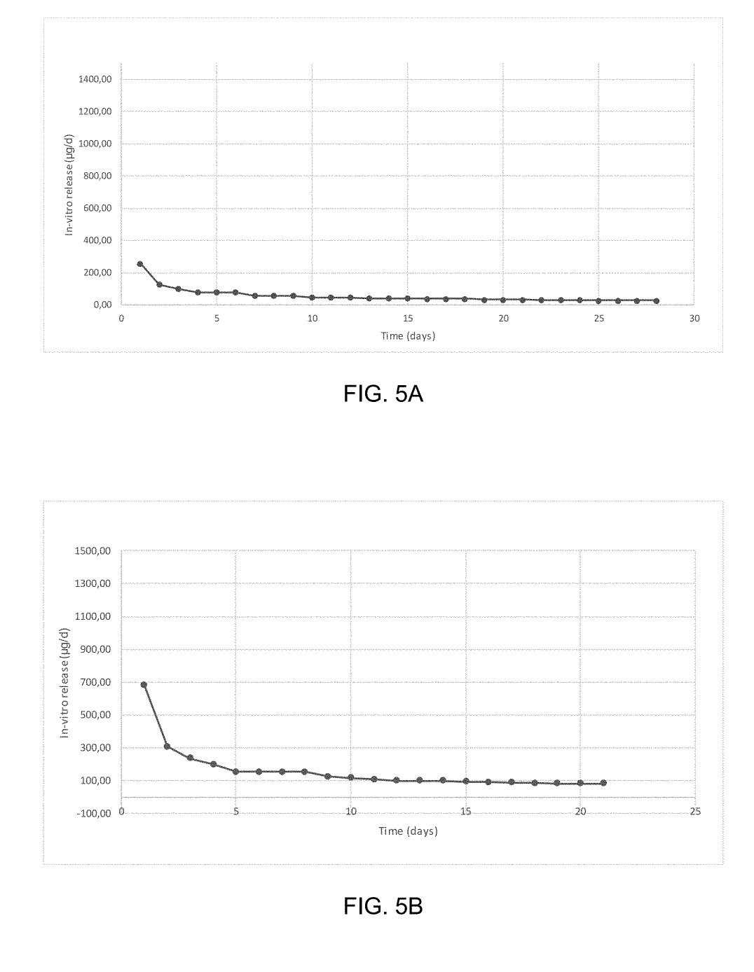

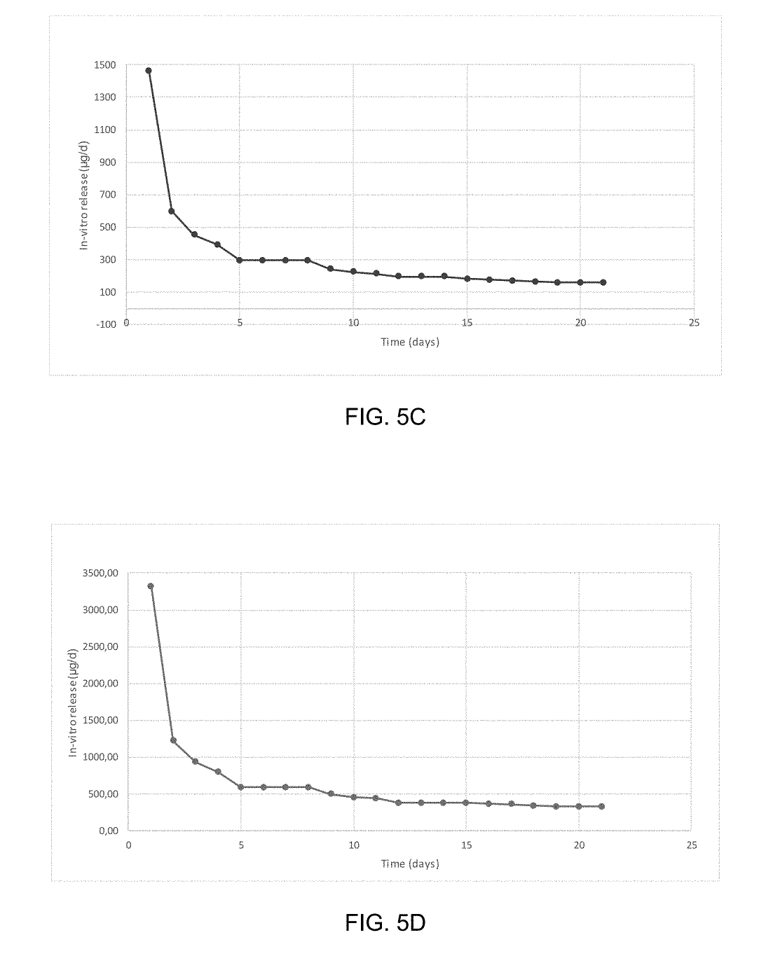

FIGS. 5A-5D show the release rate of estriol during dissolution testing of the matrix ring formed according to Example 5 for the investigated drug loadings, FIG. 5A shows the release profile for the 0.65% Estriol, FIG. 5B for the 5% Estriol loading, FIG. 5C for the 15% Estriol, and FIG. 5D for the 30% Estriol. Independent upon the loading, the release rates of these matrix type systems follow Fickian diffusion, and the diffusional exponent n ranges between 0.48 and 0.53, showing anomalous transport. All ratios of release rates d1/d21 are between 5.84 and above 10 for the tested estriol loadings. Achieving meaningful release rates of estriol from a reservoir ring is not feasible due to its physicochemical properties (low solubility in EVA with high VA contents, low permeability in EVA with low VA content).

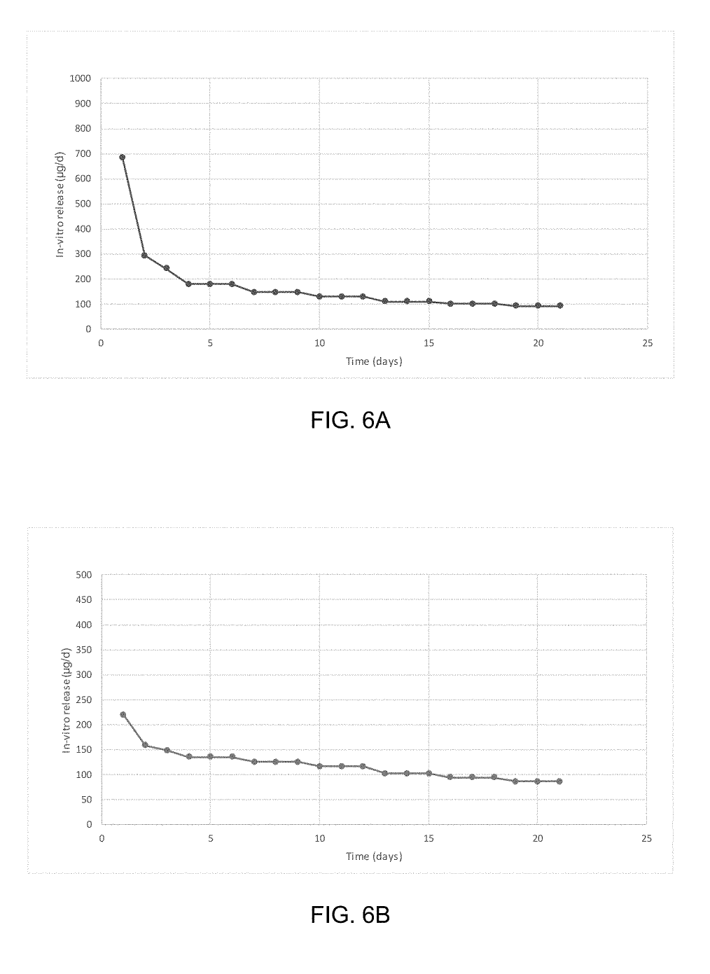

The estriol matrix system and the trimegestone reservoir system can be combined to a segmented ring as described in Example 6. FIGS. 6A and 6B show the release rates of such a segmented IVR. In FIG. 6A, the release rate of estriol from the matrix segment (30% loading; 60% segment length) is shown, FIG. 6B shows the trimegestone release from a reservoir segment (40% segment length) during dissolution testing of the segmented IVR. The release rates are proportional to the segment lengths, thereby a zero-order release is achieved for the reservoir type segment releasing trimegestone, whereas estriol is formulated into a matrix system. The ratio d1/d21 is 7.4 and 2.5 for the estriol release from the matrix and the trimegestone release from the reservoir segment, respectively.

### Example 7—Ovulation Inhibition Study of Etonogestrel/Ethinyl Estradiol (Example 2)

In a single center, open label clinical trial performed in 2 phases (pre-treatment and treatment), the vaginal ring of Example 1 was investigated in 39 women over two cycles separated by 7 treatment days. Primary efficacy parameter was ovarian activity, measured by transvaginal ultrasound according to the Hoogland and Skouby score and pituitary hormones like FSH as surrogate marker for efficacy.

Table 1 shows the Hoogland and Skouby scores obtained during clinical trials of the vaginal ring of Example 2. The results show excellent control of the follicle sizes for all women with more than 80% of women showing no or minor follicle growth (Hoogland score 1 and 2).

TABLE 1
Hoogland and Skouby scores of an etonogestrel/ethinyl estradiol vaginal ring (according to Example 2).

| | | Score | | | | | | | | | | | | | | |
|---|---|---|---|---|---|---|---|---|---|---|---|---|---|---|---|---|
| | | 6 | | 5 | | 4 | | 3 | | 2 | | 1 | | Missing | | N |
| Treatment | treatment cycle | N | [%] | N | [%] | N | [%] | N | [%] | N | [%] | N | [%] | N | [%] | total |
| Test | treatment cycle 1 | 0 | 0.00 | 0 | 0.00 | 2 | 5.13 | 0 | 0.00 | 7 | 17.95 | 30 | 76.92 | 0 | 0.00 | 39 |
| | treatment cycle 2 | 0 | 0.00 | 0 | 0.00 | 8 | 20.51 | 1 | 2.56 | 14 | 35.90 | 15 | 38.46 | 1 | 2.56 | 39 |
| | Total | 0 | 0.00 | 0 | 0.00 | 10 | 12.82 | 1 | 1.28 | 21 | 26.92 | 45 | 57.69 | 1 | 1.28 | 78 |

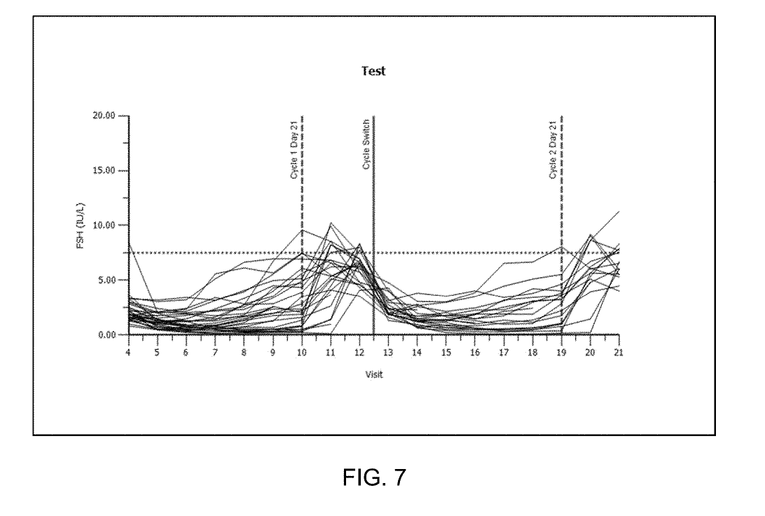

FIG. 7 shows the concentration of Follicle Stimulating Hormone (FSH) overtime during clinical trials of the vaginal ring of Example 2. The figure shows an overlay of individual profiles of FSH concentration per time point (visit) during treatment cycle 1 and 2 under treatment with the vaginal ring of Example 1 following a 21-day application+7 days treatment-free break per cycle (PPS). Insertion of the vaginal ring in the second treatment cycle is marked by a vertical red line between visit 12 and 13.

### Example 8—Mean Plasma Concentration Study of Estriol

In a single center, open-label, randomized (allocation to treatment), balanced, parallel-group trial with single dose application the following three vaginal rings were tested in postmenopausal women for 21 days.
Device 1: a vaginal ring, formed according to Example 5, having a 5% estriol loading, and having a nominal estriol delivery rate of 0.125 mg/day. The ring was administered by vaginal application in 10 women.
Device 2: a vaginal ring, formed according to Example 5, having a 15% estriol loading, and having a nominal estriol delivery rate of 0.250 mg/day. The ring was administered by vaginal application in 10 women.
Device 3: a vaginal ring, formed according to Example 5, having a 30% estriol loading, and having a nominal estriol delivery rate of 0.500 mg/day. The ring was administered by vaginal application in 10 women.

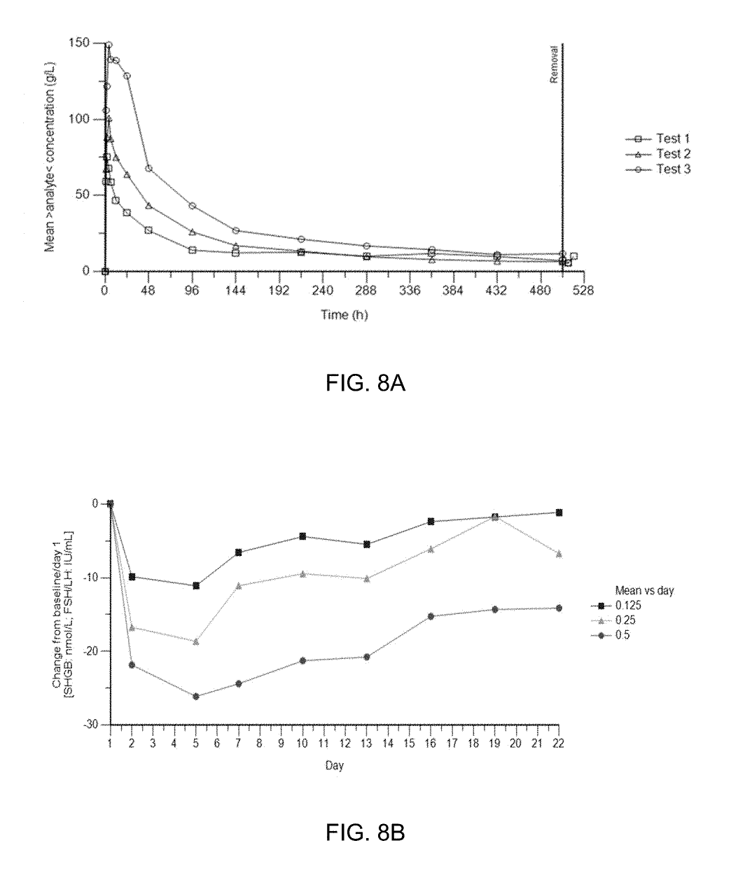

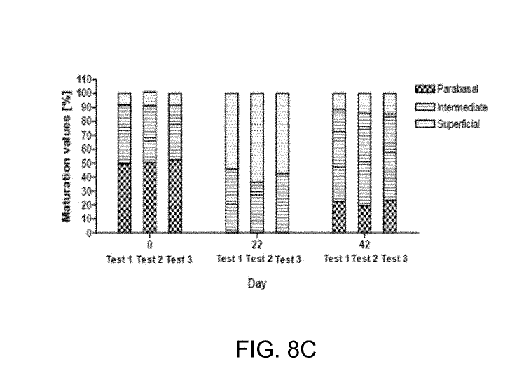

FIG. 8A depicts the mean plasma concentration vs. time curves of estriol during a single vaginal application of 1 vaginal ring of Device 1 (Test 1), Device 2 (Test 2), and Device 3 (Test 3) over 21 days.

FIG. 8B depicts the mean plasma concentration vs. time curves for changes from baseline of FSH during single vaginal application of a single vaginal ring of Device 1 (Test 1), Device 2 (Test 2), and Device 3 (Test 3) over 21 days.

FIG. 8C depicts mean maturation index by cell types (maturation values) over time during single vaginal application of 1 vaginal ring of Device 1 (Test 1), Device 2 (Test 2), and Device 3 (Test 3) over 21 days.

### Example 9—Mean Plasma Concentration Study of Estriol and Trimegestone

In a single center, open-label, randomized (allocation to treatment), balanced, parallel-group trial with single dose application the following three vaginal rings were tested in fertile women for 21 days.
Device 1: a vaginal ring, formed according to Example 6, having a 30% estriol loading, an estriol segment length of 60%, a 1.90% trimegestone core loading with a 320 um skin thickness, and having a nominal delivery rate of 0.400 mg/day for estriol and a nominal delivery rate of 0.050 mg/day for trimegestone. The ring was administered by vaginal application in 10 women.
Device 2: a vaginal ring, formed according to Example 6, having a 15% estriol loading, an estriol segment length of 60%, a 1.90% trimegestone core loading with a 195 um skin thickness, and having a nominal delivery rate of 0.300 mg/day for estriol and a nominal delivery rate of 0.095 mg/day for trimegestone, vaginal application in 10 women.
Device 3: a vaginal ring, formed according to Example 6, having a 5% estriol loading, an estriol segment length of 60%, a 1.90% trimegestone core loading with a 135 um skin thickness, and having a nominal delivery rate of 0.209 mg/day for estriol and a nominal delivery rate of 0.137 mg/day for trimegestone, vaginal application in 10 women.

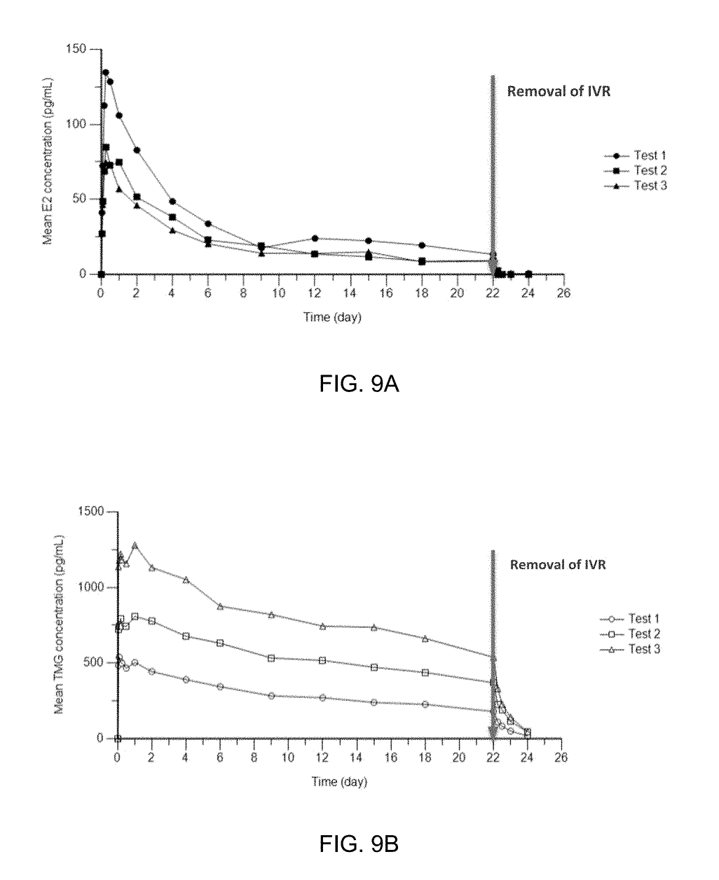

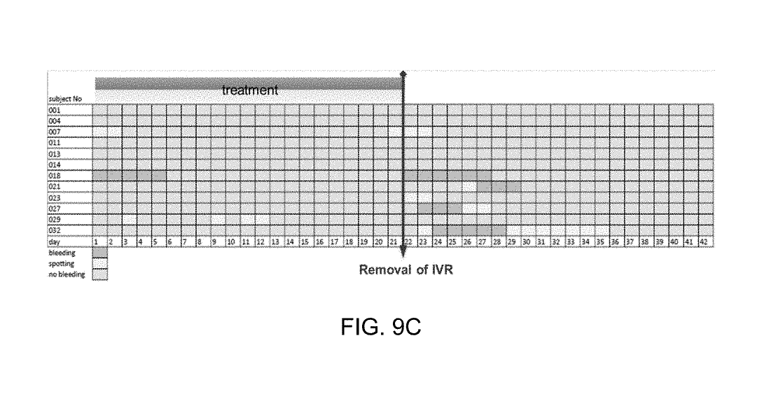

The mean trimegestone plasma levels and mean estradiol plasma levels were analyzed in women. FIG. 9A depicts the mean plasma concentration vs. time curves of estriol during a single vaginal application of 1 vaginal ring of Device 1 (Test 1), Device 2 (Test 2), and Device 3 (Test 3) over 21 days.

FIG. 9B depicts the mean plasma concentration vs. time curves of trimegestone during a single vaginal application of 1 vaginal ring of Device 1 (Test 1), Device 2 (Test 2), and Device 3 (Test 3) over 21 days.

FIG. 9C depicts the bleeding profile under treatment of Test 3 (Device 3), the vaginal ring with a delivery rate of 0.209 mg/day for estriol and 0.137 mg/day for trimegestone.

Most women under Test 3 had a good bleeding control during treatment with few bleeding and spotting episodes during treatment and a predictable initiation of bleeding after the ring has been removed.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. An intravaginal drug delivery device comprising:
one or more compartments, each of the compartments comprising an estrogen and/or a progestin dispersed in a thermoplastic polymeric matrix;
wherein the intravaginal drug delivery device provides the estrogen and/or the progestin according to a non-zero order release profile.

2. The device of claim 1, comprising:
one or more uncoated compartments, the uncoated compartments comprising an estrogen and/or progestin dispersed in an uncoated thermoplastic polymeric matrix; and/or
one or more coated compartments, the coated compartments comprising an estrogen and/or progestin dispersed in a coated thermoplastic polymeric matrix, wherein the coated thermoplastic polymeric matrix comprises a coating surrounding the coated thermoplastic polymeric matrix.

3. The device of claim 1, wherein the compartments have different sizes.

4. The device of claim 1, wherein the device comprises at least one compartment containing a progestin, and wherein the progestin is etonogestrel.

5. The device of claim 1, wherein the device comprises at least one compartment containing a progestin, and wherein the progestin is trimegestone.

6. The device of claim 5, wherein the device releases trimegestone in doses between 0.075 and 025 mg/day.

7. The device of claim 1, wherein the estrogen is ethinyl estradiol.

8. The device of claim 1, wherein the estrogen is estriol.

9. The device of claim 8, wherein the device releases estriol in doses between 0.05 and 0.75 mg/day.

10. The device of claim 8, wherein the device is configured such that estriol plasma levels of 50-200 pg/ml are achieved on day 1 of treatment

11. The device of claim 8, wherein the device is configured such that estriol plasma levels of 15-30 pg/ml are achieved on day 21 of treatment

12-14. (canceled)

15. The device of claim 1, wherein the thermoplastic matrix comprises an ethylene vinyl acetate copolymer.

16. The device of claim 1, wherein the thermoplastic matrix comprises one or more hydrophilic matrix materials.

17. The device of claim 1, wherein the thermoplastic matrix comprises an ethyl vinyl acetate copolymer and one or more hydrophilic matrix materials.

18. The device of claim 1, wherein the device has a substantially annular form.

19. The device of claim 1, wherein the device delivers an effective amount of the progestin and the estrogen for at least 21 days.

20. The device of claim 1, wherein the device comprises a progestin and an estrogen, and wherein the ratio of the release rate of the estrogen on day 1 to the release rate of the estrogen on day 21 is between 1.5 and 4.0, and wherein the ratio of the release rate of progestin on day 1 to the release rate of progestin on day 21 is between 1.5 and 4.0.

21. The device of claim 1, wherein the device comprises a progestin and an estrogen, and wherein the ratio of the release rate of the estrogen on day 1 to the release rate of the estrogen on day 21 is between 1.5 and 3.0, and wherein the ratio of the release rate of progestin on day 1 to the release rate of progestin on day 21 is between 1.5 and 3.0.

22. The device of claim 1, wherein the device comprises a progestin and an estrogen, and wherein the ratio of the release rate of the estrogen on day 1 to the release rate of the estrogen on day 21 is between 1.5 and 2.0, and wherein the ratio of the release rate of progestin on day 1 to the release rate of progestin on day 21 is between 1.5 and 2.0.

23. A method of producing a contraceptive state in a subject comprising positioning an intravaginal drug delivery device, as described in claim 1, in the vagina or uterus of a female.
